SEQUENCE LISTING (5 sequences)

SEQ ID NO: 1 - protein, 7 aa, Artificial Sequence
  Pattern in 90%  of Cecropins
QEAGLKK

SEQ ID NO: 2 - protein, 15 aa, Artificial Sequence
  Cecropin-Melittin Hybrid
  MOD_RES (15)..(15): AMIDATION
KWKLFKKIGAVLKVL

SEQ ID NO: 3 - protein, 20 aa, Artificial Sequence
  Antimicrobial peptide
FLGVVFKLASKVFPAVFGKV

SEQ ID NO: 4 - protein, 20 aa, Artificial Sequence
  Antimicrobial peptide
FLFRVASKVFPALIGKFKKK

SEQ ID NO: 5 - protein, 20 aa, Artificial Sequence
  Cecropin-Melittin hybride
FLFRVASKVFPALIGKFKKK

(19) United States
(12) Patent Application Publication
(10) Pub. No.: US 2007/0254006 A1
(43) Pub. Date: Nov. 1, 2007

(54) Medical Devices and Coatings with Non-Leaching Antimicrobial Peptides

(75) Inventors: Christopher Loose, Cambridge, MA (US); William Shannan O'Shaughnessy, Boston, MA (US); LinO Ferreira, Cambridge, MA (US); Andreas Zumbuehl, Bern (CH); Robert Langer, Newton, MA (US); Gregory Stephanopoulos, Winchester, MA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignee: Massachusetts Institute of Technology

(21) Appl. No.: 11/675,500

(22) Filed: Feb. 15, 2007

Related U.S. Application Data
(60) Provisional application No. 60/744,050, filed on Mar. 31, 2006.
(60) Provisional application No. 60/885,578, filed on Jan. 18, 2007.

(51) Int. Cl. A61K 9/70; A61F 2/00; A61K 38/00

(52) U.S. Cl. 424/423; 424/443; 514/2

(57) ABSTRACT

Antimicrobial peptides enable an alternate approach to developing antimicrobial coatings due to their targeting of the membranes of the bacteria. High specific activity is achieved by orienting the peptides so that the antimicrobial ends of the peptides maximally contact the bacteria. In one embodiment, one end of the peptide is covalently attached directly to the substrate. In another embodiment, the peptides are immobilized on the substrate using a coupling agent or tether. Non-covalent methods include coating the peptide onto the substrate or physiochemically immobilizing the peptides on the substrate using highly specific interactions, such as the biotin/avidin or streptavidin system. The compositions are substantially non-leaching, antifouling, and non-hemolytic. The immobilized peptides retain sufficient flexibility and mobility to interact with and de endocytosed by the bacteria, viruses, and/or fungi upon exposure. Immobilizing the peptides to the substrate reduces concerns regarding toxicity of the peptides and the development of antimicrobial resistance, while presenting substantially all of the peptide at the site of action at the surface of the substrate.

### RELATED APPLICATION

This application claims priority to U.S. Ser. No. 60/774,050, which was filed on Feb. 15, 2006, U.S. Ser. No. 11/561,266, which was filed on Nov. 17, 2006, and U.S. Ser. No. 60/885,578, which was filed on Jan. 18, 2007.

### FIELD OF THE INVENTION

The present invention is generally in the field of immobilized bioactive peptide coatings, specifically peptide coatings which exhibit bacteriostatic and bacteriocidal properties.

### BACKGROUND OF THE INVENTION

Hospital infections are becoming increasingly costly and difficult to treat due to the spread of drug resistant bacteria. Despite efforts to improve the sterility of surgical procedures, infection remains common. These infections are often associated with medical devices. Skin penetrating devices, such as central venous catheters, as well as urinary catheters, provide a route for bacteria to enter the body and implanted devices form favorable surfaces on which bacteria can grow.

Once bacteria colonize a medical device, they may form a recalcitrant biofilm. A biofilm is a complex aggregation of microorganisms marked by the excretion of a protective and adhesive matrix. Biofilms are also often characterized by surface attachment, structural heterogeneity, genetic diversity, complex community interactions, and an extracellular matrix of polymeric substances. The biofilm protects bacteria in the interior of the film from the immune system. Systemic antibiotics are ineffective in treating such infections due to their limited ability to penetrate biofilms. For these reasons, the treatment of device infections often involves the removal of the device, administration of antibiotics, followed by the insertion of a new device. This procedure may be costly and painful, and if the bacteria are not completely cleared, the new device may become infected.

A variety of controlled-release antimicrobial coatings and devices have been developed, particularly for devices such as central venous catheters (CVCs) and wound dressings, for which bacterial infection is especially problematic. Existing antimicrobial coatings generally consist of antibiotic agents or metal ions incorporated into the device surface or polymer coating. Slow release of these agents results in localized toxic concentrations that help reduce bacterial colonization and proliferation.

There are currently three antimicrobial CVCs with significant clinical use. ARROWg+ard® Blue catheters (Arrow International) are impregnated with a combination of chlorhexidine (Kuyyakanond, et al., FEMS Micro. Let., 100(1-3), 211-215 (1992)) and silver sulfadiazine, whose antimicrobial activity is primarily due to silver's disruption of the electron transport chain and DNA replication (Silver et al., J. Ind. Micro. Biotech., 33(7), 627-634 (2006); Fox et al., Antimicrob. Agents & Chemotherapy, 5(6), 582-588 (1974). These catheters have been shown in clinical studies to reduce catheter colonization by 44% (Veenstra et al., J. Amer. Med. Assoc., 281 (3), 261-267 (1999)). Chlorhexidine, however, is known to result in hypersensitivity reactions in patients (Wu et al., Biomaterials, 27(11):2450-67 (2006)), and both chlorhexidine and silver sulfadiazine may induce bacterial resistance (Brooks et al., Inf. Con. Hos. Epidem., 23(11): 692-695 (2002); Silver et al., J. Ind. Micro. Biotech., 33(7), 627-634 (2006)).

Cook Critical Care's Spectrum® line of catheters utilizes the slow release of minocycline, which disrupts protein synthesis (Speers et al., Clin. Microbio. Rev., 5(4): 387-399 (1992)) and rifampin, which inhibits RNA polymerase (Kim et al., Sys. Appl. Microbiol., 28(5): 398-404 (2005)). These catheters have been shown in clinical studies to reduce catheter colonization by 69% (Raad et al., Ann. Int. Med., 127(4): 267 (1997)). However, minocycline and rifampin are also known to induce bacterial resistance (Kim et al, Sys. Appl. Microbiol., 28(5): 398-404 (2005); Speers et al, Clin. Microbio. Rev., 5(4): 387-399 (1992)).

Edwards Lifesciences' Vantex® catheters release silver, carbon, and platinum ions, with most of the antimicrobial activity attributed to the silver ions. These catheters have a demonstrated reduction in catheter colonization of approximately 35%, which may be limited in part by the in vivo sequestration of silver ions by albumin in the blood stream (Ranucci et al., Crit. Care med., 31(1): 52-59 (2003); Corral et al., J. Hos. Infec., 55(3): 212-219 (2003)). Bacterial resistance to silver ions has also been reported (silver et al., J. Ind. Micro. Biotech., 33(7), 627-634 (2006)).

A number of antimicrobial would dressings have also been developed, with the majority based on the incorporation of silver ions, such as ConvaTec's Aquacel®. Other antimicrobial agents include cadexomer iodine (Smith & Nephew's Iodoflex™ and Iodosorb™), CHG (Johnson & Johnson's Biopatch™), and PHMB (Kendall Healthcare's Kerlix™ AMD™.)

An attractive alternative to these agents are antimicrobial peptides (AmPs). AmPs can distinguish between mammalian cells and microbes based on membrane properties, and kill microbes using a fast and non-specific mechanism of attack. This mechanism is thought to be dramatically less likely to induce drug resistance as compared to antibiotics that target specific enzymes because the evolutionary cost for changing membrane properties is greater and the attack is sufficiently fast that bacteria have little opportunity to survive and mutate. Naturally occurring AmPs may have activity against Gram positive and negative bacteria, fungi, viruses, and even cancerous cells (Jenssen, Hamill et al., Clin Microbiol Rev., 19 (3); 491-511 (2006)).

It has been shown that releasing AmPs from the surface of a device has the ability to prevent device related infections. Simply soaking a Dacron graft in a solution of the AmP dermaseptin before implanting it in a rat and challenging with bacteria, reduces the incidence of device colonization and infection (Balaban et al., Antimicrob. Agents & Chemother., 48: 2544-2550 (2004)). The release of dermaseptin was effective against both methicillin resistant and vancomycin intermediate-resistant Staphylococcus aureus. Migenix and Cadence's antimicrobial peptide drug candidate CPI-226 has shown in vivo efficacy in a slow release cream formulation in clinical trails against bacteria associated with medical device infection.

Slow release coatings suffer from several inherent limitations. By design, slow-release coatings have a limited lifespan. For many catheter applications, including CVCs and dialysis catheters, extended protection is desired by clinicians. Additionally, slowly released antibiotics create neighboring regions of sub-lethal drug concentrations that may encourage the development of drug resistance. By releasing drugs into the bloodstream, there are also increased concerns over systemic toxicity. Finally, due to the large loading of drug that may be required to create a slow release coating, the structural and performance properties of the device may be impacted.

U.S. Patent Application Publication Nos. 2005/0065072 to Keeler et al. and 2004/0126409 to Wilcox et al., and European patent No. EP 0 990 924 to Wilcox et al. describe coupling antimicrobial peptides to a variety of substrates to provide antimicrobial devices. However, the coupling methods are random, so that there is no control over the orientation of the peptides on the surface. The coupling methods immobilize the peptide to the substrate via any amine on the peptide, including those within basic side chains frequently found in antimicrobial peptides. Thus, the AmPs may be tethered at a number of different sites, or one molecule may be tethered at multiple positions. While this will not prevent the surface from being bactericidal, the efficacy will not be as great as if peptides are positioned on the surface of a material so that the orientation and flexiblity of the peptides are optimal to maximize the antimicrobial activity per amount of peptide, potentially lowering cost and toxicity.

It is therefore and object of the present invention to provide a material having antimicrobial peptides coupled thereto with enhanced efficacy in preventing microbial attachment and proliferation.

### SUMMARY OF THE INVENTION

Compositions containing one or more types of antimicrobial peptides immobilized on a substrate with a specific orientation, and methods of making and using thereof, are described herein. Antimicrobial peptides enable an alternate approach to developing antimicrobial coatings due to their targeting of the membranes of the bacteria. Unlike most traditional antibiotics, which must be released to reach their targets in the interior of bacterial cells, most AmPs must only contact the outer membrane or cell wall of the bacteria to be effective. Peptides which are immobilized using the methods described herein have a higher specific antimicrobial activity as compared to the same peptides randomly attached to a substrate.

The peptides can be immobilized on the substrate using covalent or non-covalent methods. High specific antimicrobial activity is achieved by orienting the peptides to enable effective interaction between critical portions of the AmP and the bacterial membrane. In one embodiment, one end of the peptide is covalently attached directly to the substrate. In another embodiment, the peptides are immobilized on the substrate using a coupling agent or tether. Suitable coupling agents include small organic molecules, polymers, and combinations thereof. In another embodiment, the peptides are immobilized on a polymeric thin film which has been applied to the substrate. In still another embodiment, the peptide is immobilized to a polymer which is covalently attached to the substrate. For example, the peptides can be immobilized on polymer brushes, dendrimeric polymers, or crosslinked polymers forming a hydrogel attached to a substrate. Non-covalent methods include coating the peptide onto the substrate or physiochemically immobilizing the peptides on the substrate using highly specific interactions, such as the biotin/avidin or streptavidin system. The peptides can be tethered in a desired density and orientation using chemistries designed to reduce protein adhesion. In one embodiment, the tether contains hydrophilic groups to reduce protein adhesion. In another embodiment the substrate is modified with a hydrophilic polymer to which the AmPs can subsequently be tethered. This hydrophilic polymer can be either covalently tethered to the substrate or compose a conformal coating of the substrate. In a preferred embodiment, the peptides are bound to the substrate at a concentration of at least 0.001, 0.01, 0.1, 0.25, 0.5, 1, 1.5, 2.5, 5, 10, 25 or 50 mg peptide/cm³ substrate surface area.

The peptides can be coated onto a variety of different types of substrates including medical implants such as vascular grafts, orthopedic devices, dialysis access grafts, and catheters; surgical tools, surgical garments; and bandages. The substrates can be composed of metallic materials, ceramics, polymers, fibers, inert materials such as silicon, and combinations thereof. The compositions described herein are substantially non-leaching, antifouling, and non-hemolytic. The immobilized peptides retain sufficient flexibility and mobility to interact with the bacteria, viruses, and/or fungi upon exposure to the peptides. Immobilizing the peptides to the substrate reduces concerns regarding toxicity of the peptides and the development of antimicrobial resistance, while presenting large peptide concentrations at the site of action at the surface of the substrate.

### BRIEF DESCRIPTION OF THE DRAWINGS

FIG. 1 shows an AmP immobilized on the surface of a substrate via a hydrophilic tether.

FIG. 2 shows hydrophilic tethers, with and without AmP coupled thereto, immobilized on the surface of a substrate.

FIG. 3 shows AmPs immobilized on a hydrogel which is immobilized on the surface of a substrate.

FIG. 4 shows a schematic of amidated polymer brushes coupled to a vinyl presenting substrate.

FIG. 5 shows the structure of N-(γ-maleimidobutyryloxy) sulfosuccinimide ester (sulfo-GMBS). The sulfonated N-hydroxysuccinimide residue reacts with primary amines while the maleimido group reacts with thiol groups.

### DETAILED DESCRIPTION OF THE INVENTION

I. Definitions

“Amino acid residue” and “peptide residue”, as used herein, refer to an amino acid or peptide molecule without the —OH of its carboxy group (C-terminally linked) or one proton of its amino group (N-terminally linked). In general the abbreviations used herein for designating the amino acids and the protective groups are based on recommendations of the IUPAC-IUB Commission on Biochemical Nomenclature (see Biochemistry (1972) 11:1726-1732). Amino acid residues in peptides are abbreviated as follows: alanine is Ala or A; Cysteine is Cys or C; Aspartic Acid is Asp or D; Glutamic Acid is Glu or E; Phenylalanine is Phe or F; Glycine is Gly or G; Histidine is His or H; Isoleucine is Ile or I; Lysine is Lys or K; Leucine is Leu or L; Methionine is Met or M; Asparagine is Asn or N; Proline is Pro or P; Glutamine is Gln or Q; Arginine is Arg or Ar; Serine is Ser or S; Threonine is Thr or T; Valine is Val or V; Tryptophan is Trp or W; and Tyrosine is Tyr or Y. Formylmethionine is abbreviated as fMet or Fm. By the term “residue” is meant a radical derived from the corresponding α-amino acid by eliminating the OH portion of the carboxy group and one of the protons of the α-amino group. The term “amino acid side chain” is that part of an amino acid exclusive of the —CH(NH₂)COOH backbone, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or —H (the side chain of glycine).

“Non-naturally occurring amino acid”, as used herein, refers to any amino acid that is not found in nature. Non-natural amino acids include any D-amino acids (described below), amino acids with side chains that are not found in nature, and peptidomimetics. Examples of peptidomimetics include, but are not limited to, β-peptides, γ-peptides, and δ-peptides; oligomers having backbones which can adopt helical or sheet conformations, such as compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination. All of the amino acids in the human body, except glycine, are either right-hand or left-hand versions of the same molecule, meaning that in some amino acids the positions of the carboxyl group and the R-group are switched. Nearly all of the amino acids occurring in nature are the left-hand versions of the molecules, or the L-forms. Right-hand versions (D-forms) are not found in the proteins of higher organisms, but they are present in some lower forms of life, such as in the cell walls of bacteria. They also are found in some antibiotics, among them, streptomycin, actinomycin, bacitracin, and tetracycline. These antibiotics can kill bacterial cells by interfering with the formation of proteins necessary for viability and reproduction.

“Polypeptide”, “peptide”, and “oligopeptide” refers generally to peptides and proteins having more than about ten amino acids, preferably more than 9 and less than 150, more preferably less than 100, most preferably between 9 and 51 amino acids. The polypeptides can be “exogenous,” meaning that they are “heterologous,” i.e., foreign to the host cell being utilized, such as human polypeptide produced by a bacterial cell. Exogenous also refers to substances that are added from outside of the cells, not endogenous (produced by the cells). A peptide encompasses organic compounds composed of amino acids, whether natural or synthetic, and linked together chemically by peptide bonds. The peptide bond involves a single covalent link between the carboxyl (oxygen-bearing carbon) of one amino acid and the amino nitrogen of a second amino acid. Small peptides with fewer than about ten constituent amino acids are typically called oligopeptides, and peptides with more than ten amino acids are termed polypeptides. Compounds with molecular weights of more than 10,000 Daltons (50-100 amino acids) are usually termed proteins.

“Antimicrobial peptide” (“AmP”), as used herein, refers to oligopeptides, polypeptides, or peptidomimetics that kill (i.e., bacteriocidal) or inhibit the growth of (i.e., bacteriostatic) microorganisms including bacteria, yeast, fungi, mycoplasma, viruses or virus infected cells, and/or protozoa. In some instances, AmPs have been reported to have anticancer activity. Generally antimicrobial peptides are cationic molecules with spatially separated hydrophobic and charged regions. Exemplary antimicrobial peptides include linear peptides that form an α-helical structure in membranes or peptides that form β-sheet structures optionally stabilized with disulfide bridges in membranes. Representative antimicrobial peptides include, but are not limited to, cathelicidins, defensisn, dermeidin, and more specifically magainin 2, protegrin, protegrin-1, melittin, 11-37, dermaseptin 01, cecropin, caerin, ovispirin, and alamethicin. Naturally occurring antimicrobial peptides include peptides from vertebrates and non-vertebrates, including plants, humans, fungi, microbes, and insects.

“Immobilization” or “immobilized”, as used herein, refers to an antimicrobial peptide that is attached to a substrate. The peptide can be attached covalently or non-covalently to the substrate, such that is substantially non-leaching. Immobilized peptides retain sufficient flexibility and mobility to interact with bacteria, viruses, and/or fungi upon exposure.

“Antimicrobial” as used herein, refers to molecules that kill (i.e., bactericidal) or inhibit the growth of (i.e., bacteriostatic) microorganisms including bacteria, yeast, fungi, mycoplasma, viruses or virus infected cells, cancerous cells, and/or protozoa. More specifically, “bactericidal” as used herein, refers to molecules that kill microorganisms including bacteria, yeast, fungi, mycoplasma, viruses or virus infected cells, and/or protozoa.

“Immobilized antimicrobial”, as used herein, refers to surfaces having antimicrobial peptides immobilized thereon that kill (i.e., bactericidal) or inhibit the growth of (i.e. bacteriostatic) microorganisms including bacteria, yeast, fungi, mycoplasma, viruses or virus infected cells, and/or protozoa that come into contact with the surface. More specifically, “immobilized bactericidal activity” as used herein, refers to the reduction in viable microorganisms including bacteria, yeast, fungi, mycoplasma, viruses or virus infected cells, and/or protozoa that contact the surface. For bacterial targets, bactericidal activity may be quantified as the reduction of viable bacteria based on the ASTM 2149 assay for immobilized antimicrobials, which may be scaled down for small samples as follows: an overnight culture of a target bacteria in a growth medium such as Cation Adjusted Mueller Hinton Broth, is diluted to approximately 1×10⁵ cfu/ml in pH 7.4 Phosphate Buffered Saline using a predetermined calibration between OD₆₀₀ and cell density. A 0.5 cm² sample of immobilized antimicrobial surface is added to 0.75 ml of the bacterial suspension. The sample should be covered by the liquid and should be incubated at 37° C. with a sufficient amount of mixing that the solid surface is seen to rotate through the liquid. After 1 hour of incubation, serial dilutions of the bacterial suspension are plated on agar plates and allowed to grow overnight for quantifying the viable cell concentration. Preferably at least a 1 log reduction in bacterial count occurs relative to a control of bacteria in phosphate buffered saline (PBS) without a solid sample. More preferably, at least a 2 log reduction in bacteria count occurs. Even more preferably, at least a 3 log reduction in bacteria count occurs. Most preferably, at least a 4 log reduction in bacteria count occurs.

“Substantially non-leaching”, as used herein, means that the compositions do not leach a sufficient amount of the antimicrobial peptide in the presence of pH 7.4 Phosphate Buffered Saline to demonstrate solution antimicrobial properties or generate a toxic reaction in a host from the released material. Activity from released material may be evaluated by performing the immobilized antimicrobial assay described above, removing the supernatant liquid, and centrifuging out the remaining bacteria. The supernatant may then be inoculated with bacteria to yield a 1×10⁵ cfu/mL suspension, held for one hour at 37° C., and dilutions and plating carried out to quantify the concentration of viable cells. Preferably, a 10%, 20%, or 50% reduction in viable cells does not occur over the course of 1 hour, 3 hours, 1 day, 3 days, 7 days, or 30 days. More preferably, the composition does not release a sufficient amount of the antimicrobial peptide in the presence of blood, tissue, and/or in an in vivo setting to demonstrate solution antimicrobial properties or generate a toxic reaction in a host over the course of 1 hour, 1 day, 3 days, 7 days, or 30 days. In one embodiment, the composition doe snot release more than 10 μg/cm² of peptide, preferably not more than 1 μg/cm² of peptide, in the defined time period.

“”, as used herein, refers to the non-covalent attachment of a protein, cell, or other substance to a surface. The amount of adhered substance may be quantified by sonicating and/or rinsing the surface with an appropriate resuspension agent such as Tween or SDS, and quantifying the amount of substance resuspended.

“Substantially Cytotoxic”, as used herein, refers to a composition that changes the metabolism, proliferation, or viability of mammalian cells that contact the surface of the composition. This may be quantified by the International Standard ISO 10993-5 which defines three main tests to assess the cytotoxicity of materials including the extract test, the direct contact test and the indirect contact test.

“A substantially non-hemolytic surface”, as used herein, means that the composition does not lyse 50%, 20%, 10%, 5%, or most preferably 1%, of human red blood cells when the following assay is applied: A stock of 10%, washed pooled red blood cells (Rockland Immunochemicals Inc., Gilbertsville, Pa.) is diluted to 0.25% with a hemolysis buffer of 150 mM NaCl and 10 mM Tris at pH 7.0. A 0.5 cm2 antimicrobial sample is incubated with 0.75 ml of 0.25% red blood cell suspension for 1 hour at 37° C. The solid sample is removed and cells spun down at 6000 g, the supernatant removed, and the OD414 measured on a spectrophotometer. Total hemolysis is defined by diluting 10% of washed pooled red blood cells to 0.25% in sterile DI water and incubating for 1 hour at 37° C., and 0% hemolysis is defined by a suspension of 0.25% red blood cells in hemolysis buffer without a solid sample.

“Substantially non-fouling”, as used herein, means that the composition reduces the amount of adhesion of proteins, including blood proteins, plasma, tissue and/or bacteria to the substrate relative to the amount of adhesion to a reference polymer such as polyurethane. Preferably, a device surface will be substantially non-fouling in the presence of human blood. Preferably the amount of adhesion will be decreased 20%, 50%, 75%, 90%, 95%, or most preferably 99%, relative to the reference polymer.

“Substantially non-toxic”, as used herein, means a surface that is substantially non-hemolytic and substantially non-cytotoxic.

“Biocompatibility”, as used herein, refers to a surface that is substantially non-toxic and non-immunogenic. More broadly, biocompatibility is the ability of a material to perform with an appropriate host response in a specific situation (Williams, D. F. Definitions in Biomaterials. In: Proceedings of a consensus Conference of the European Society for Biomaterials, Elsevier: Amsterdam, 1987). Therefore, biocompatibility represents a global statement on how well body tissues interact with a material and how this interaction meets the designed expectation for a certain implantation purpose and site (Von Recum, A. F.; Jenkins, M. E.; von Recum, H. A. Introduction: biomaterials and Biocompatibility. In: Handbook of Biomaterials Evaluation: Scientific, Technical and Clinical Testing of Implant Materials. von Recum, A. F., Ed.; Taylor & Francis, 1999, pp. 1-8). hence, biocompatibility is a relative rather than an absolute concept, which depends to a large degree on the ultimate application of the material.

“Density”, as used herein, refers to the mass of peptide that is covalently linked per surface area of substrate.

“Effective surface concentration”, as used herein, means the density of immobilized peptide sufficient to produce a desired antimicrobial response.

“Orientation”, as used herein, means that the peptide is immobilized on the surface of the substrate in such a manner that the portion of the peptide presented in interact with bacteria, viruses, and/or fungi upon exposure is uniform for all immobilized molecules of a given peptide. In addition, the amino acid residue within the peptide through which it is immobilized is controlled through selection of the coupling chemistry such that the peptide is uniformly tethered by that residue. “Uniformly” means that more than 70%, preferably more than 90%, preferably more than 95%, most preferably more than 99% of the peptide is tethered by that residue. Ideally, the oriented peptide will be attached by a single amino acid residue. However, it will be recognized by one skilled in the art that multiple attachment residues could be included within the same region of the peptide without affect the “orientation” of the attachment. Typically, the N-terminus of the peptide should be presented to target cells for highest activity, although this may vary depending on the peptide.

“Substrate”, as used herein, refers to the material on which the peptide is immobilized. The peptide may be immobilized directly to the substrate or may be coupled to the substrate using a coupling agent. Alternatively, the substrate may be coated with a thin film, membrane or gel and the peptide immobilized on the thin film, membrane or gel.

“Cysteine”, as used herein, refers to the amino acid cysteine or a synthetic analogue thereof, wherein the analogue contains a free sulfhydryl group.

“Coating”, as used herein, refers to any temporary, semipermanent or permanent layer, treating or covering or surface. The coating may be a chemical modification of the underlying substrate or may involve the addition of new materials to the surface of the substrate. It includes any addition in thickness to the substrate or change in surface chemical composition of the substrate. A coating can be a gas, vapor, liquid, paste, semi-solid or solid. In addition a coating can be applied as a liquid and solidified into a bard coating. Examples of coatings include polishes, surface cleaners, caulks, adhesives, finishes, paints waxes, polymerizable compositions (including phenolic resins, silicone polymers, chlorinated rubbers, coal tar and epoxy combinations, epoxy resin, polyamide resins, vinyl resins, elastomers, acrylate polymers, fluoropolymers, polyesters and polyurethanes, and latexes).

“Tether” or “tethering agent”, as used herein, refers to any molecule used to covalently immobilize peptide on a material where the molecule remains as part of the final chemical composition.

“Coupling agent”, as used herein, refers to any molecule or chemical substance which activates a chemical moiety, either on the peptide or on the material to which it will be attached, to allow for formation of a covalent bond between the peptide wherein the material does not remaining in the final composition after attachment.

II. Compositions

The AmPs can be applied to, immobilized on, or incorporated into a substrate using a variety of covalent and non-covalent procedures known in the art.

Suitable covalent procedures include, but are not limited to, grating or coating a polymer to the surface of a substrate to create reactive functional groups for coupling to the peptides and direct attachment of the peptides to the substrate surface. In a preferred embodiment, the coupling reaction between the peptide and the substrate involves a terminal thiol group in the antimicrobial peptide such that the peptide is oriented on the surface of the medical device. Coupling may be performed through direct reaction, use of a coupling agent, and/or use of a tethering agent. Suitable non-covalent procedures include, but are not limited to, physiochemically immobilizing the peptides on the substrate using highly specific interactions, such as the biotin/avidin or streptavidin system.

A. Substrates

The peptides may be applied to, absorbed into, or coupled to, a variety of different substrates. Examples of suitable materials include metallic materials, ceramics, polymers, fibers, inert materials such as silicon, and combinations thereof.

Suitable metallic materials include, but are not limited to, metals and alloys based on titanium (such as nitinol, nickel titanium alloys, thermo-memory alloy materials), stainless steel, tantalum, nickel-chrome, or certain cobalt alloys including cobalt-chromium-nickel alloys such as ELGILOY® and PHYNOX®.

Suitable ceramic materials include, but are not limited to, oxides, carbides, or nitrides of the transition elements such as titanium oxides, hafnium oxides, iridium oxides, chromium oxides, aluminum oxides, and zirconium oxides. Silicon based materials, such as silica, may also be used.

Suitable polymeric materials include, but are not limited to, styrene and substituted styrenes, ethylene, propylene, poly(uretane)s, acrylates and methacrylates, acrylamides and methacrylamides, polyesters, polysiloxanes. polyethers, poly(orthoester), poly(carbonates), poly(hydroxyalkanoate)s, copolymers thereof, and combinations thereof.

Substrates may be in the form of, or form part of, films, particles (nanoparticles, microparticles, or millimeter diameter beads), fibers (wound dressings, bandages, gauze, tape, pads, sponges, including woven and nonwoven sponges and those designed specifically for dental or ophthalmic surgeries), sensors, pacemaker leads, catheters, stents, contact lenses, bone implants (hip replacements, pins, rivets, plates, bone cement, etc.), or tissue regeneration or cell culture devices, or other medical devices used within or in contact with the body.
1. Effective Surface Area

In addition to the chemical composition of the substrate, the micro and nano structure of the substrate surface is important in order to maximize the surface area available for peptide attachment. For metallic and ceramic substrates, increased surface area can be created through surface roughening, for example by a random process such as plasma etching. Alternatively, the surface can be modified by controlled nano-patterning using photolithography. Polymeric substrates can also be roughened as with metallic and ceramic substrates. In addition, the surface area available for peptide attachment on a polymeric substrate can be increased by controlling the morphology of the polymer itself. Examples of this approach include polymer brushes, dendrimeric polymers, self assembling block copolymers, and shape-memory polymers.

B. Peptides

Any peptide which exhibits antimicrobial properties when immobilized to a substrate can be used in the compositions and methods described herein. Not all peptides have activity when immobilized, so it is essential to verify activity after immobilization. Methods and systems for generating peptides which exhibit antimicrobial activity when immobilized, are described in U.S. Patent Application Publication No. 2006/0035281 to Stephanopoulos et al. For example, the pattern Q.EAG.L.K.K. (SEQ ID NO: 1) (where “.” is a wildcard, indicating that any amino acid will suffice at that position in the pattern) is present in over 90% of cecropins, an AmP common in insects. Computational tools, such as TEIRESIAS can be used to produce libraries of peptides that exhibit antimicrobial activity. The peptides preferably show limited homology to naturally-occurring proteins but have strong bacteriostatic activity against several species of bacteria, including S. aureus and B. anthracis. The peptides can be synthesized using conventional methods, such as Fmoc chemistry. Once made, the designed proteins and peptides may be experimentally evaluated and tested for structure, function and stability, as required, using routine methods known to those skilled in the art. Suitable peptides are described in Wang, Z and G Wang, APD: the Antimicrobial peptide Database, Nucleic Acids Research, 2004, Vol. 32, Database issue D590-D592 and include, but are not limited to, Cecropin-Melittin Hybrid (KWKLFKKIGAVLKVL-amidated) (SEQ ID NO: 2), Cecropin P1, Temporin A, D28, D51, dermaseptin, RIP, and combinations thereof.

peptidomimetics, which exhibit antibacterial activity, may also be used. Peptidomimetics, as used herein, refers to molecules which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials can be classified into the following categories: α-peptides, β-peptides, γ-peptides, and δ-peptides. Copolymers of these peptides can also be used.

Examples of α-peptides peptidomimetics include, but are not limited to, N,N′-linked oligoureas, oligopyrrolinones, oxazolidin-2-ones, azatides and azapeptides.

Examples of β-peptides include, but are not limited to, β-peptide foladmers, α-amioxy acids, sulfur-containing β-peptide analogues, and hydrazino peptides.

Examples of γ-peptides include, but are not limited to, γ-peptide foladmers, oligoureas, oligocarbamates, and phosphodiesters.

Examples of δ-peptides include, but are not limited to, alkene-based δ-amino acids and carbopeptoids, such as pyranose-based carbopeptoids and furanose-based carbopeptoids.

Another class of peptidomimetics includes oligomers having backbones which can adopt helical or sheet conformations. Example of such compounds include, but are not limited to, compounds having backbones utilizing bipyridine segments, compounds having backbones utilizing solvophobic interactions, compounds having backbones utilizing side chain interactions, compounds having backbones utilizing hydrogen bonding interactions, and compounds having backbones utilizing metal coordination.

Examples of compounds containing backbones utilizing bipyridine segments include, but are not limited to, oligo(pyridine-pyrimidines), oligo(pyridine-pyrimidines) with hydrazal linkers, and pyridine-pyridazines.

Examples of compounds containing backbones utilizing solvophobic interactions include, but are not limited to, oligoguanidines, aedamers (structures which take advantage of the stacking properties of aromatic electron donor-acceptor interactions of covalently linked subunits) such as oligomers containing 1,4,5,8-naphthalene-tetracarboxylic diimide rings and 1,5-dialkoxynaphthalene rings, and cyclophanes such as substituted N-benzyl phenylpyridinium cyclophanes.

Examples of compounds containing backbones utilizing side chain interactions include, but are not limited to, oligothiophenes such as olihothiophenes with chiral p-phenyl-oxazoline side chains, and oligo(m-phenylene-ethynylene)s.

Examples of compound containing backbones utilizing hydrogen bonding interactions include, but are not limited to, aromatic amide backbones such as oligo(acylated 2,2′-bipyridine-3,3′-diamine)s and oligo(2,5-bis[2-aminophenyl]pyrazine)s, diaminopyridine backbones templated by cyanurate, and phenylene-pyridine-pyrimidine ethynylene backbones templated by isophthalic acid.

Examples of compounds containing backbones utilizing metal coordination include, but are not limited to, zinc bilinones, oligopyridines complexed with Co(II), Co(III), Cu(II), Ni(II), Pd(II), Cr(III), or Y(III), oligo(m-phenylene ethynylene)s containing metal-coordinating cyano groups, and hexapyrrins.

In one embodiment, the peptide is the antimicrobial peptide D28 (FLGVVFKLASKVFPAVFGKV) (SEQ ID NO:3) and/or D51 (FLFRVASKVFPALIGKFKKK) (SEQ ID NO:4). In another embodiment, the peptide is a quorum sensing inhibitor such as RNA-III inhibiting peptide (RIP) that is either slowly released from the coating or is covalently tethered in a manner that enables its biofilm-inhibition activity. In yet another embodiment, the peptide is a combination of one or more AmPs and/or RIPs.

The peptides can be provided in solution, suspension, or immobilized, as discussed below. The peptides may be chemically modified, for example, by pegylation using commercially available reagents and methods, in order to prolong in vivo half-life and inhibit uptake by the reticuloendothelial system (RES). The peptides can also be coupled to one or more other proteins, lipids, or compounds.

The antimicrobial peptides should be active when coupled to the substrate. Preferentially, the orientation of the peptide and nature of the tether is designed to maximize antimicrobial activity for a peptide sequence and density. The peptides should be oriented in such a way that the active region of the peptide is available to interact with bacteria, viruses, and/or fungi. For example, the peptides can be designed so that a cysteine residue is located in a particular position in order to orient the peptide so that the active end of the peptide can interact with bacteria, viruses, and/or fungi upon exposure.

The compositions are highly active, exhibit broad spectrum activity, and are substantially non-hemolytic. The compositions are preferably antifouling; that is, the compositions inhibit protein adhesion which can decrease the efficacy of the antimicrobial peptides. This may be accomplished by the use of a coupling agent or tether with antifouling properties to couple the peptide to the substrate. Preferentially, the compositions should also release the bacteria from the substrate upon killing so that the surface is reusable to treat future infections.

C. Tethers, Linkers and Spacers

Tethers, linkers and spacers are utilized both for attachment of peptides to substrates and or attachment of peptides to polymer films coated on substrates. The tether composition can be varied according to the surface chemistry of the substrate or the polymer covalently attached to, or coated onto, the substrate. Tether length and composition can be varied to optimize peptide interaction with bacteria encountering the surface and to maximize the antifouling properties of the surface. The composition must also be selected such that the peptide retains the correct orientation when presented on the surface so as to have biological activity. Preferably, the tether should form a non-leaching surface. Specific tethers are discussed below with respect to the various coupling methods.

D. Hydrophilic Polymers

The production of anti-fouling surfaces is a key element in the development of biomedical materials, such as medical devices and implants. Such coatings limit the interactions between the implants and physiological fluids. Different approaches can be adopted to create surfaces that have non-fouling properties, including the use of hydrophilic tethers, hydrophilic polymers or hydrogels covalently attached to the substrate.
1. Hydrophilic Tethers

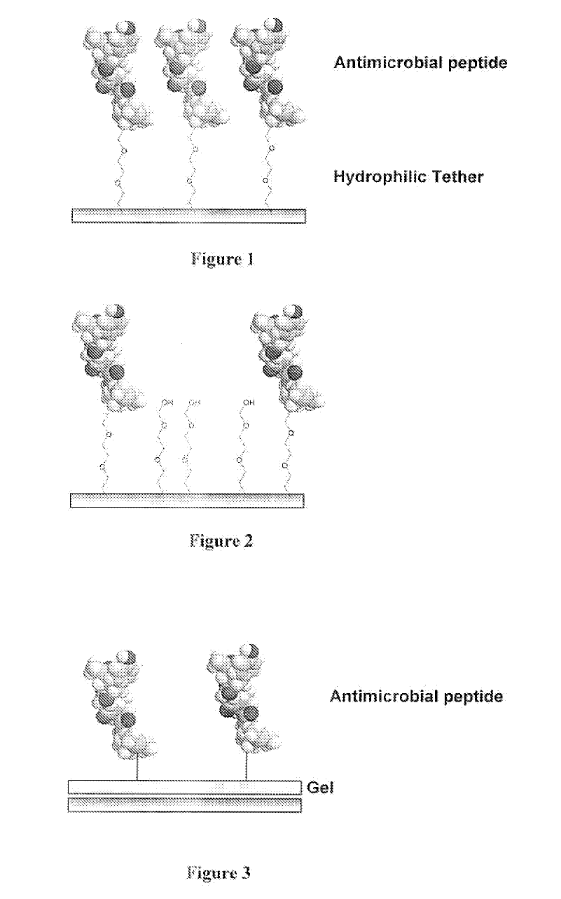

In one embodiment, the tether contains a hydrophilic polymer, such as poly(ethylene glycol) (PEG). FIG. 1 shows a peptide immobilized to the surface of a substrate via PEG. The number of repeat units in the polymer can vary from 4-100, most preferably 4-16. PEG has been demonstrated to create non-fouling surfaces (Michel et al., Langmuir 2005, 12327-12332). Optimized tether length and composition are functions of both substrate composition and the particular peptide being tethered. Multi-arm PEGs can be used to increase the number of functional groups for antimicrobial peptide immobilization.

In another embodiment, the tether is a polysaccharide such as dextran, hyaluronic acid, chitin, chitosan, starch, cellulose, insulin, alginate, agarose, xanthan. In a preferential embodiment, the polysaccharide is dextran. Dextran surface coatings are capable of limiting protein and cell adhesion. It has been demonstrated that dextran monolayers are very effective in reducing BSA adsorption to silver surfaces and that the effect was dependent on the surface coverage by dextran but not on the thickness of the monolayer (Frazier et al. Biomaterials 2000, 21, 957-966). Furthermore, Österberg et al. (J. Biomed. Mat. Res. 1995, 29, 741-747) showed that dextran bound to aminated polystyrene surfaces was able to reduce fibrinogen adhesion and was even more effective than PEG. It has also been demonstrated that protein adsorption appears to be insensitive to polymer layer thickness. Dextran compares favorably to PEG as a tether since more hydroxyl groups are available for the immobilization of antimicrobial peptides.
2. Separate Immobilized Hydrophilic Polymers

To create non-fouling surfaces, hydrophilic polymers which are not tethered to peptide can be immobilized on the substrate. FIG. 2 shows PEG covalently attached to the substrate surface. In this case, the hydrophilic polymer is not a tether but acting as a non-fouling agent. The hydrophilic polymers described in the section above can be used for this purpose.
3. Hydrogels

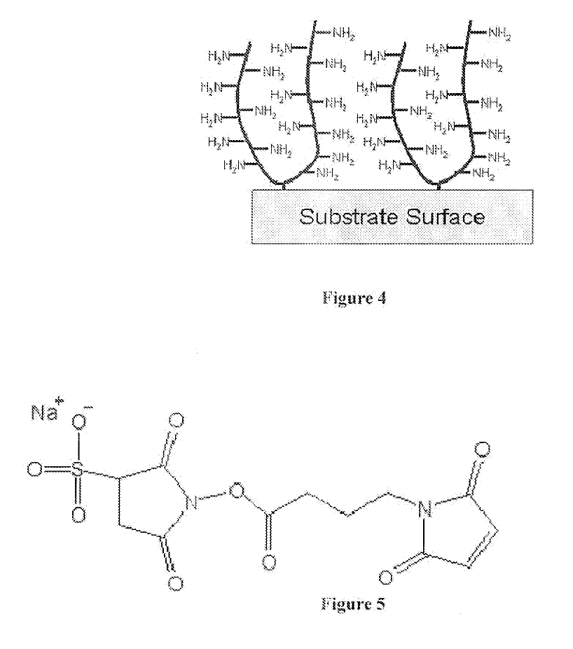

hydrogels can be used as non-fouling coatings on the substrate, or can be used as the substrate itself. FIG. 3 shows AmPs immobilized on a hydrogel, which is coated onto the substrate. In a preferred embodiment, anti-microbial peptides can be immobilized on the surface of the hydrogel. Hydrogels are three-dimensional, hydrophilic, polymeric networks capable of imbibing large amounts of water or biological fluids (Peppas et al. Eur. J. Pharm. Biopharm. 2000, 50, 27-46). These networks are composed of homopolymers or copolymers, and are insoluble due to the presence of chemical crosslinks or physical crosslinks, such as entanglements or crystallites. Hydrogels can be classified as neutral or ionic, based in the nature of the side groups. In addition, they can be amorphous, semicrystalline, hydrogen-bonded structures, supermolecular structures and hydrocollodial aggregates (Peppas, N. A. Hydrogels. In: Biomaterials science: an introduction to materials in medicine; Ratner, B. D., Hoffman, A. S., Schoen, F. J., Lemons, J. E., Eds; Academic Press, 1996, pp. 60-64; Peppas et al. Eur. J. Pharm. Biopharm. 2000, 50, 27-46). Hydrogels can be prepared from synthetic or natural monomers or polymers.

Medical devices can be coated with hydrogels using a variety of techniques, examples of which include spraying, dipping, and brush coating. A small quantity of gel solution (e.g., in the microliter range) can be used to treat a surface area of 1 cm². The amount of gel solution per unit area and the corresponding coating solution concentration and application rate can be readily determined for any particular application.

Hydrogels can be prepared from synthetic polymers such as poly(acrylic acid) and its derivatives [e.g. poly(hydroxyethyl methacrylate) (pHEMA)], poly(N-isopropylacrylamide), poly(ehtylene glycol) (PEG) and its copolymers and poly(vinyl alcohol) (PVA), among others (Bell, C. L.; Peppas, N. A. Adv. Polym. Sci. 1995, 122, 125-175; Peppas et al. Eur. J. Pharm. Biopharm. 2000, 50, 27-46; Lee, K. Y.; Mooney, D. J. Chem. Rev. 2001, 101, 1869-1879.). Hydrogels prepared from synthetic polymers are in general non-degradable in physiologic conditions. Hydrogels can also be prepared from natural polymers including but not limited to, polysaccharides, proteins, and peptides. These networks are in general degraded in physiological conditions by chemical or enzymatic means.

In one embodiment, the hydrogel is non-degradable under relevant in vitro and in vivo conditions. Stable hydrogel coatings are necessary for certain applications including central venous catheters coating, heart valves, pacemakers and stents coatings. In other cases, hydrogel degradation may be a preferential approach such as in tissue engineering constructs.

In a preferred embodiment, the gel is formed by dextran. Dextran is a bacterial polysaccharide, consisting essentially of α-1,6 linked D-glucopyranose residues with a few percent of α-1,2, α-1,3, or α-1,4-linked side chains. Dextran is widely used for biomedical applications due to its biocompatibility, low toxicity, relatively low cost, and simple modification. This polysaccharide has been used clinically for more than five decades as a plasma volume expander, peripheral flow promoter and antithrombolytic agent (Mehvar, R. J. Control. Release 2000, 69, 1-25). Furthermore, it has been used as macromolecular carrier for delivery of drugs and proteins, primarily to increase the longevity of therapeutic agents in the circulation. Dextran can be modified with vinyl groups either by using chemical or enzymatic means to prepare gels (Ferreira et al. Biomaterials 2002, 23, 3957-3967).

Dextran-based hydrogels can be considered as non-fouling materials. Dextran-based hydrogels prevent the adhesion of vascular endothelial, smooth muscle cells, and fibroblasts (Massia, S. P.; Stark, J. J. Biomed. Mater. Res. 2001, 56, 390-399. Ferreira et al. 2004, J. Biomed. Mater. Res. 68A, 584-596) and dextran surfaces prevent protein adsorption (Österberg et al. J. Biomed. Mat. Res. 1995, 29, 741-747).

E. Polymer Microstructure

As discussed above, the maximum possible surface loading of AmP can be increased through the creation of microstructure on the substrate surface. For polymeric substrates, including hydrogel networks, this surface morphology can be created through appropriate polymer structural design, such as dendrimers and brush copolymers. One example of this is the growth of surface tethered dedrimeric polymers. Poly(amidoamine) (PAMAM) dendrimer can be grown from an amine presenting surface through alternating reactions of methyl acrylate and ethylene diamine (Nguyen et al. Langmuir, 2006, 22, 7825-7832). Each generation of dendrimer added effectively doubles the number of sites available for peptide attachment. In addition, when synthesis is terminated after an amidation step, the resulting material is an amine presenting polymer that may behave as an anti-fouling hydrogel, similar to poly(ethylene glycol) (Champman et al. Langmuir, 2001, 17, 1225-1233).

Another example of tailoring polymer microstructure to increase AmP surface loading is the growth of polymer brushes from the substrate surface. These are polymer chains tethered at one end to the substrate but extending from the substrate into the surrounding medium. This approach creates many additional sites for peptide attachment, the number of which depends on the molecular weight of the brush polymer. One such system is brush growth of poly(methyl acrylate) (PMA). Following polymerization of even moderate molecular weight PMA the material can be functionalized, leading to the surface presentation of 50-100 times more AmP than that possible through direct surface attachment. A schematic showing an amidate brush covalently linked to a substrate surface is shown in FIG. 4.

F. Other Active Agents

In addition to the antimicrobial peptides, one or more therapeutic, prophylactic or diagnostic agents, which can be proteins or small organic or inorganic molecules, may be coupled to the substrate. In one embodiment, the substrate includes a bioactive agent which is released independently of the immobilized bioactive peptides.

For example, agents which inhibit encapsulation, scarring, and/or cell proliferation may be immobilized with the antimicrobial peptide on the substrate. Other examples of bioactive molecules include antiproliferative, cytostatic or cytotoxic chemotherapeutic agents, antimicrobial agents, antiinflammatories, growth factors, and cell adhesion peptides.

In another embodiment, one or more agents are tethered to the substrate using a hydrolyzable linkage so that the agent is slowly released from the substrate, for example, at the site of implantation or insertion of a medical device.

Alternatively, one or more agents are non-covalently associated with the surface. For example, one or more agents can be entrapped within a hydrogel material and released by diffusion and/or degradation of the hydrogel material.

II. Methods for Immobilizing Antimicrobial Peptides

Unlike traditional antibiotics which must diffuse into target cells, the AmPs may retain antimicrobial activity when tethered, covalently or non-covalently, to a substrate. When immobilized, the portion of the AmP available to interact with bacteria may affect the antimicrobial activity of the surface. This is a major reason why orientation of the peptides is important to enhance specific activity of the peptides.

A number of methods such as those described below can be used to create the required functional moieties for AmP tethering on a variety of surfaces. The density of the attachment groups affects the density of the attached peptides. Tethers, which can vary in branching, length of branches, and chemical nature of branches can be sued to decrease protein adherence or increase AmP loading while presenting AmPs in a manner that is bactericidal.

In addition to the density of attachment, peptide orientation is an important factor in the bioactivity of the immobilized AmPs. Oriented peptide attachment can be achieved by a number of synthetic approaches. One approach s to incorporate into the peptide an amino acid residue containing a chemical moiety otherwise not present in the peptide. Cystine, containing a thiol group, is one example. If no other cysteine residue is present in the peptide, the addition of this residue, and its functional moiety, will create a chemically unique location in the peptide sequence. This location can then be utilized, through appropriate coupling chemistry, for the oriented immobilization of the peptide on a surface. At a pH from about 7 to about 8.5, the free thiol group is deprotonated to form a strong nucelophile, in contrast to amine groups on amino acids which are deprotonated at higher pH. Thus, one can selectively couple the thiol group of the cysteine residue with the substrate or tether by controlling the pH of the reaction conditions.

It should be noted that while this additional residue will most preferably be included at either the C-terminus or N-terminus of the peptide, oriented attachment can also be achieved if the unique residue occurs anywhere in the peptide sequence. It will also be obvious to one skilled in the art that multiple copies of the same residue placed together at the desired locus of attachment would effect the same “oriented attachment” as a single such residue. Other approaches for oriented attachment of an AmP include, but are not limited to: functionalization of either the N or C-terminus of the peptide with a reactive moiety not naturally present in peptides (such as an epoxide ring) for selective use in tethering the peptide to the surface and/or protection of all copies of a given moiety (using appropriate protecting groups such as Fmoc chemistry) except those at the location of desired attachment followed by reaction of the unprotected groups for attachment and then deprotection.

Covalent Procedures for Coupling Peptides to a Substrate
1. Direct Attachment of the Peptide to the Substrate Surface

Coupling of the Peptide to the Substrate without a Coupling Agent or Tether

In one embodiment, the AmP is coupled directly to the substrate surface. The chemistry used to couple the AmP to the substrate depends on the chemical composition of the substrate surface. The substrate surface can be treated in a variety of ways known in the art to introduce the desired functional group(s). Surface modification can be accomplished through gas-phase techniques including, but not limited to, plasma, corona discharge, flame treatment, UV/ozone, UV and ozone only, or wet chemistry including, but not limited to, aminolysis, hydrolysis, reduction, activation of alcohol chain ends with tosyl chloride and subsequently chemistry, graft copolymerisation of vinyl compounds by chemical initiation, and ion beam treatment in the presence of vinyl monomers. For example, the substrate surface can be treated with a plasma, microwave, and/or corona source to introduce hydroxyl, amine, and/or carboxylic acid groups to the substrate surface, which can react with functional groups on the peptide.

The antimicrobial peptides can be immobilized directly on the substrate through their thiol groups, in an oriented way. This can be achieved through a variety of methods. First, thiol groups in the antimicrobial peptide can react directly with unsaturated groups on the substrate such as maleimides (Schelté et al., Biocon. Chem., 11, 118-123 (2000)), vinyl sulfones (Masri et al., J. Protein Chem., 1988, 7, 49-54; Morpurgo et al., Biocon. Chem., 7, 363-368 (1996)), acrylamides (Romanowska et al., Meth. Enzym., 242, 90-101 (1994)) and acrylates (Lutolf et al., Biocon. Chem., 12, 1051-1056 (2001)) present in the surface of medical devices by conjugate addition reaction (also termed Michael type addition reaction). This reaction can be carried out at physiological temperature and physiological pH (pH 7.4) and was shown to be selective versus biological amines (Elbert et al., J. Controlled Release 2001, 76, 11-25; Lutolf et al., Biomacromolecules, 4, 713-722 (2003)). Second, thiol groups in the antimicrobial peptide can react with epoxide functional groups in the substrate surface. Thiol groups are highly reactive nucleophiles with epoxides, requiring a buffered system in the pH range of 7.5-8.5 for efficient coupling.

In another embodiment, the antimicrobial peptides may be bound covalently to a device surface by any functional group (e.g., amine, carbonyl, carboxyl, aldehyde, alcohol) present in the peptide. For example, one or more amine or alcohol or thiol groups on the antimicrobial peptide may be reacted directly with isothiocyanate, acyl azide, N-hydroxysuccinimide ester, aldehyde, epoxide, anhydride, lactone, or other functional groups incorporated onto the surface of the device. Schiff bases formed between the amine groups on the peptide and aldehyde groups of the device can be reduced with agents such as sodium cyanoborohydride to form hydrolytically stable amine links (Ferreira et al., J. Molecular Catalysis B: Enzymatic 2003, 21, 189-199). Alternatively, the free amino or hydroxyl groups of the antimicrobial peptides are attached to a surface containing epoxide functional groups. The reaction of the epoxide functional groups with hydroxyls requires high pH conditions, usually in the pH range of 11-12. Amine nucleophiles react at a more moderate alkaline pH values, typically needing buffer environments of at least pH 9.

Coupling of the Peptide to the Substrate by a Coupling Agent

The antimicrobial peptide can be coupled directly to the substrate by the use of a reagent or reaction that activates a group on the surface of the substrate or the antimicrobial peptide making it reactive with a functional group on the peptide or substrate, respectively, without the incorporation of a coupling agent. In general, the immobilization of the antimicrobial peptide is non-oriented. For example, carbodiimides mediate the formation of amide linkages between a carboxylate and an amine or phosphoramidate linkages between phosphate and an amine. Examples of carbodiimides are 1-ethyl-3-(3-dimethylaminopropyl)carbodiimide hydrochloride (EDC), 1-cyclohexyl-3-(2-morpholinoethyl)carbodiimide (CMC), dicyclohexyl carbodiimide (DCC), diisopropyl carbodiimide (DIC), and N,N′carbonyldiimidazole (CDI). N-ethyl-3-phenylisoxazolium-3′-sulfonate (Woodward's reagent) mediates the formation of amide linkages though the condensation of carboxylates and amines. CDI can also be sued to couple amino groups to hydroxyl groups.

In one embodiment, a device surface containing terminal carboyxl groups is activated with EDC in buffer pH 5.0 for 15-30 minutes and then the activated surface reacted with the peptide for 2-3 h in PBS, pH 7.4, at room temperature.

Coupling of the Peptide to the Substrate using a Tether

The coupling of the peptide to the substrate may also be accomplished using a tether. The tether may have terminal functionalities that react with surface-amine and peptide-sulfhydryl groups. In this case, the antimicrobial peptide is immobilized into the surface in an oriented way. These tethers may contain a variable number of atoms. Examples of tethers include, but are not limited to, N-succinimidyl 3-(2-pyridyldithio)propionate (SPDP, 3- and 7-atom spacer), long-chain-SPDP (12-atom spacer), (Succinimidyloxycarbonyl-α-methyl-2-(2-pyridyldithio)toluene) (SMPT, 8-atom spacer), Succinimidyl-4-(N-maleimidomethyl)cyclohexane-1-carboxylate (SMCC, 11-atom spacer) and Sulfosuccinimidyl-4-(N-maleimidomethyl)cyclohexane-1-carboxylate, (sulfo-SMCC, 11-atom spacer), m-Maleimidobenzyol-N hydroxysuccinimide ester (MBS, 9-atom spacer), N-(γ-maleimidobutyryloxy)succinimide ester (GMBS, 8-atom spacer), N-(γ-maleimidobutyryloxy)sulfosuccinimide ester (sulfo-GMBS, 8-atom spacer), Succinimidyl 6-((iodoacetyl)amino)hexanoate (SIAX, 9-atom spacer), Succinimidyl 6-(6-(((4-iodoacetyl)amino)hexanoyl)amino)hexanoate (SIAXX, 16-atom spacer), and p-nitrophenyl iodoacetate (NPIA, 2-atom spacer). One ordinarily skilled in the art also will recognize that a number of other coupling agents, with different number of atoms, may be used. In a preferential embodiment, the succinimide group of sulfo-GMBS is reacted with the amine groups from the substrate surface. In a subsequent step, the terminal maleimide group from sulfo-GMBS is reacted with sulfhydryl groups from the peptide. The structure of sulfo-GMBS is shown in FIG. 5.

Moreover, spacer molecules may be incorporated into the tether to increase the distance between the reactive functional groups at the termini. For example, polyethylene glycol (PEG) can be incorporated into sulfo-GMBS. Hydrophilic molecules such as PEG have also been shown to decrease biofouling of surfaces when covalently coupled.

In certain embodiments, the free amine groups of the antimicrobial peptide are attached to a surface containing reactive hydroxyl groups, in a non-oriented way. As an example, N,N′-Carbonyldiimidazole (CDI) can activate the hydroxyl groups of the surface with the concomitant formation of an imidazole carbamate. This reaction must take place in nonaqueous environments (e.g., acetone, dimethyl sulfoxide (DMSO), tetrahydrofuran (THF), dimethylformamide (DMF)) with less than 1% water due to the rapid breakdown of CDI by hydrolysis. Finally, the activated surface can react with an amine-containing peptide solubilized in a buffer with pH between 7 and 10 (Ferreira et al., J. Molecular Catalysis B: Enzymatic 2003, 21, 189-199).

In other embodiments, the free amine groups of the antimicrobial peptide are attached to a surface containing reactive amine groups. Again, using this chemistry there is no control in peptide orientation. Tethers such as dithiobis(succinimidylpropionate) (DSP, 8atom spacer), disuccinimidyl suberate (DSS, 8-atom spacer), glutaraldehyde (4-atom spacer), Bis[2-succinimidyloxycarbonyloxy)ethyl]sulfone (BSOCOES, 9-atom spacer) and others that one skilled in the art also will recognize, can be used for this purpose.

In another embodiment, the tether may contain identical functional groups at each end that react with functional groups on the substrate and the peptide. For example, a homobifunctional tether is first reacted with a thiol surface in aqueous solution (for example PBS pH 7.4) and then in a second step the peptide is coupled to the tether. Examples of homobifunctional sulfhydryl-reactive tethers include, but are not limited to, 1,4-Di-[3′-2′-pyridyldithio)propion-amido]butane (DPDPB, 16-atom spacer) and Bismaleimidohexane (BMH, 14-atom spacer). This specific chemistry allows one to control the orientation of the peptide.

The choice of concentration of the tether utilized for activity will vary as a function of the volume, agent and substrate chosen for a given application, as will be appreciated by one skilled in the art.

Following peptide immobilization, the surface may be washed with water or phosphate buffer saline or other buffer to remove unreacted antimicrobial peptide and solvent. The buffer may contain small amounts of a surfactant (e.g., Sodium dodecyl sulfate, Tween®, Triton®) to facilitate the removal of the antimicrobial peptide that is not covalently immobilized. The removal of the peptide can be monitored by HPLC or by commercial kits used to quantify peptides and proteins (e.g. BCA kit from Sigma).
2. Grafting Polymers to a Substrate

In another embodiment, a polymer is grafted onto a substrate and the AmP is covalently coupled to the polymer. The polymer is chosen based on the desired functional group to be used to couple the AmP to the substrate. Examples of suitable functional groups on the polymer include, but are not limited to, amines, carboxylic acids, epoxides, and aldehydes. In another embodiment, reactive monomers containing the desired functional groups can be polymerized on a substrate using techniques such as chemical vapor deposition (CVD).
Polymer Growth in Solution or from the Surface of the Substrate

Polymers can be grafted to a substrate using a variety of techniques known in the art. For example, the polymer can be grown in solution and then coupled to the surface of the substrate. Alternatively, the polymer can be grown from the substrate surface. The polymer can be grown in solution or from the substrate using a variety of polymerization techniques including, but not limited to, free radical polymerization, anionic polymerization, cationic polymerization, and enzymatic polymerization. Polymers grown from the substrate can also be prepared using dendrimer synthesis. Examples of free-radical polymerization include spontaneous UV polymerization; type 1 or type 2 UV initiated polymerization; thermal initiated polymerization using a thermal initiator, such as AIBN; or redox-pair initiated polymerization. In the case of the polymers grown from the surface of the substrate, the surface is typically functionalized with the same moiety used for polymerization (e.g., vinyl groups for free radical polymerization).

Suitable polymers include, but are not limited to, poly(lactone), poly(anhydride), poly(urethane), poly(orthoester), poly(ethers), poly(esters), poly(phosphazine), poly(ether ester)s, poly(amino acids), synthetic poly(amino acids), poly(carbonates), poly(hydroxyalkanoate)s, polysaccharides, cellulosic polymers, proteins, such as zein, modified zein, casein, gelatin, gluten, serum albumin, collagen, actin, -fetoprotein, globulin, macroglobulin, cohesion, laminin, fibronectin, fibrinogen, ostelcalcin, osteopontin, osteoprotegerin, and blends and copolymers thereof.

In one embodiment, a cysteine-incorporating Cecropin-Melittin hybride peptide (KWKLFKKIGAVLKVLC-NH₂) (SEQ ID NO: 5), KWKLFKKIGAVLKVLC-amidated (SEQ ID NO:5), with a single point of attachment at the C, was immobilized on amidated polymer brushes coupled to a substrate. The polymer brushes were prepared by polymerizing the brush monomer aminoethyl methacrylate in the presence of a vinyl presenting substrate. The peptide was immobilized using sulfo-GMBS chemistry.

Polymer brushes can also be attached to materials such as silicone or polyurethane, which are commonly used to make catheters. As described above, the growth of polymer brushes typically requires the presence of vinyl moieties on the substrate. In order to introduce vinyl groups onto the surface of silicone substrates, the silicone can be treated with a pure oxygen plasma followed by emersion in ethanol to create a surface that is purely hydroxyl in nature. Following hydroxylation, the surface can be exposed to an evaporated vinyl silane, such as trichlorovinyl silane or trimethoxy-vinyl silane. The vinylated substrate can then be sued to attach brush polymers. Polyurethane substrates can be treated in an analogous manner using a plasma treatment with CO₂, O₂, ammonia. The resulting hydroxyl and/or amine groups can be acrylated to form vinyl moieties on the surface followed by tethering of the polymer brushes. Polymer brushes typically have reactive functional groups, such as amines, at surface concentrations 10-100 times higher than those possible through direct surface functionalization. The increased flexibility of polymer brushes may also help to decrease biofouling.
Chemical Vapor Deposition

Monomers can be polymerized on a substrate using techniques such as chemical vapor deposition. Chemical vapor deposition (CVD) is a process by which a thin film is deposited directly from the gas phase onto a substrate. Films having a thickness less than 100 nm can be applied to substrates of any size, shape, composition, and complexity. The polymer can be deposited using plasma/microwave CVD, hot filament CVD, initiated CVD, and photo-initiated CVD. In one embodiment, a polymerizable monomer and a free radical initiator are fed simultaneously into a CVD reaction chamber containing a hot filament to form a thin polymer film of controlled chemistry. Within the chamber, the radical initiator is activated by a resistively heated filament. The resulting radicals react with monomer molecules which have absorbed onto the substrate surface to form the thin polymer film. CVD can be used to coat substrates of all shapes and almost any composition with a high degree of conformation. The filament temperature required to activate the initiator is mild enough to avoid damage to the monomer species, allowing for the retention of reactive functional groups within the resulting film. In addition, the substrate temperature can be independently controlled allowing further tailoring of the film properties as well as deposition on a wide range of substrates. CVD coatings may be deposited at very mild substrate conditions (e.g. substrate held at room temperature) in order to deposit coatings onto delicate substrates, such as tissue paper. The polymerizable monomer is chosen based on the desired functional group used to couple the polymer to the peptide.

The coated substrates are prepared by placing the substrate, such as a silicon wafer, into the CVD reactor. A functionalized monomer, such as GMA, and a free radical initiator, such as tert-amyl peroxide, are introduced into the reactor. The flow rates of the functionalized monomer and the initiator, as well as the filament temperature and the substrate temperature, can be independently controlled to achieve the desired thickness of the thin film. Monomer flow is generally in the range of 1-50 sccm, with the initial flow between 1:1 and 1:20 versus the monomer. To ensure uniform deposition, total flow to the reactor should be scaled such that no more than 10% of the monomer is reacted before leaving the deposition chamber. The initiator is decomposed by the filament at a temperature of 180-650° C. Initiation can also be performed by plasma or pulsed plasma at a power of 5-200 W. Film deposition of 1-200 nm/min has been demonstrated, though rates are typically between 5-50 nm/min. Other examples of initiating species that can be utilized for CVD deposition of these and other monomers include, but are not limited to: tert-butyl peroxide, azo-t-butane, and other azo or peroxide compounds with vapor pressure such that a flowrate of >0.1 sccm can be established into the vacuum reactor. After deposition, the chemical composition of the deposited films can be verified using IR spectroscopy. The final density of the tethered AmP can be controlled by varying the surface density of the functional groups on the monomers. For example, styrene, which does not contain an epoxy functional group, may be titrated with GMA to produce films with the desired density of attachment sites.

Suitable functionalized monomers include, but are not limited to, glycidyl methacrylate (“GMA”), which contains reactive epoxide groups, aminoethyl methacrylate, and ethylene imine.
Peptide Attachment

methods for tethering peptides to CVD coated surface under conditions that do not damage sensitive substrates have been described in Murthy et al., Langmuir, 20, 4774-4776 (2004).

Following deposition of the polymer film, the functional groups on the polymer are activated for peptide attachment. For example, the epoxide groups on pGMA can be reacted with hexamethylene diamine in ethanol for 5 hours at 60° C. in a sealed glass vial to generate free amines. The free amines can react with a carboxylic acid group on the AMP to immobilize the AMP to the substrate. In one embodiment, a commercially available gluteraldehyde kit (Polysciences) is used to link the carboxylic acid group of the AMP to the free amine on the substrate. The flexibility of the tethered AMP can be optimized by varying the length of the covalent tether. In the case of the gluteraldehyde tether, the chain is 12 carbon atoms long (include the free amine and the carboxyl group). Additional flexibility can be provided, for example, by adding glycine residues at the tethered end of the peptide between the functional sequence and the glutamic acid tethering group. Glycine buffer lengths of 0, 4, 8, and 12 amino acids can be added to achieve the desired flexibility. The surface density of the peptides can be mapped by labeling the peptides with a stable fluorochrome and evaluating the surface using fluorescent microscopy.

Peptides can be coupled to polymers grown in solution and coupled to the substrate or grown from the surface of the substrate using the same chemistries described above for directly coupling peptides to the substrate surface.

B. Physiochemical Methods for coupling Peptides to Substrates

The antimicrobial peptide may be bound physically to a substrate or device. Suitable physiochemical methods for immobilizing peptides to a substrate include highly specific interactions such as the biotin/avidin or streptavidin system.
1. Biotin/Avidin or Streptavidin

Biotin and its derivatives, including, but not limited to, NHS-biotin, sulf-NHS-biotin, 1-biotinamido-4-[4′-(maleimidomethyl)cyclohexane-carboxamido]butane (Biotin-BMCC), N-iodoacetyl-N-biotinylhexylenediamine, cis-tetrahydro-2-oxothieno[3,4-d]-imidazoline-4-valeric acid hydrazide, can be covalently incorporated into antimicrobial peptides through an amine (Hofmann et al., PNAS 1977, 74, 2697-2700; Gretch et al., Anal. Biochem. 1987, 163, 270-377), sulfhydryl (Sutoh et al., J. Mol. Biol. 1984, 178, 323-339), carbonyl or carboxyl groups (O'Shannessy et al., Immunol. Lett. 1984, 8, 273-277; Rosenberg et al., J. Neurochemistry 1986, 46, 641-648) present in the peptide. The biotinylation of antimicrobial peptide favors its orientation when immobilized in the device surface. Biotin's interaction with the proteins avidin and streptavidin is among the strongest noncovalent affinities known (Kₐ=10¹⁵ M⁻¹).
2. Polyhistidine-Nickel Chelate Coupling

Stable complexes can be formed by reacting polyhistidine tags with chelated nickel cations including, but not limited to, Ni²⁺ tridentate or Ni²⁺ nitrilotriacetic acid. In one embodiment, the matrix can be derivatized with a polyhistidine tag ligand which can form a complex with a Ni²⁺ tridentate or nitrilotriacetic-derivatized biomolecule.
3. Salicylhydroxamic Acids

Reagents suitable for the modification of the substrate for the purpose of attaching a salicylhydroxamic acid moiety for subsequent conjugation/complexation to one or more peptides having pendant phenyl boronic acid groups have the general formula shown below: 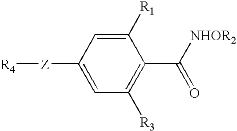
wherein R₄ is a reactive electrophilic or nucleophilic moiety suitable for reaction of the salicylhydroxamic acid molecule with the matrix material or R₄ is a moiety capable of reacting in a redox process, e.g. the formation of a disulfide bond. R₂ is an H, an alkyl, or a methylene or ethylene moiety with an electronegative substituent. R₁ and R₃ are independently H or hydroxy and Z is optionally a spacer molecule comprising a saturated or unsaturated chain from 0 to 6 carbon equivalents in length, an unbranched or branched, saturated or unsaturated chain from 6 to 18 carbon equivalents in length with at least one intermediate amine or disulfide moiety, or a polyethylene glycol chain of 3-12 carbon equivalents in length. In one embodiment, the salicylhydroxamic acid ligand is attached to the surface through the agent salicylhydroxylamine hydrazide. In other embodiments, the salicylhydroxamic acid ligand can be attached to the surface with a salicylhydroxylamine N-hydroxysuccinimide (“NHS”) ester or carboxylic acid.
4. Phenyl Boronic Acids

Phenyl boronic acid reagents, many of which are known in the art, can be appended to the antimicrobial peptide to afford a conjugate having one or more pendant phenyl boronic acid moieties as shown below:

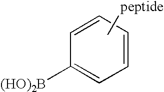

The reagent may include a group comprising a spacer molecule such as an aliphatic chain up to 6 carbon equivalents in length, an unbranched aliphatic chain of 6 to 18 carbon equivalents in length with at least one intermediate amide or disulfide moiety, or a polyethylene oxide or polyethylene glycol chain of 3-12 carbon equivalents in length. The use of spacer molecules such as polyethylene oxide and polyethylene glycol may allow for higher mobility of the peptide in aqueous solution. The peptide may also include a portion of a reactive moiety used to attach the peptide to the phenyl boronic acid species in the absence of a spacer molecule. The phenyl boronic acid species can comprise one, two, or three boronic acid groups attached in various positions about the aromatic ring.

III. Methods of Use

The materials described above maybe in the form of a medical device to which the antimicrobial peptide is applied as a coating. Suitable devices include, but are not limited to, surgical, medical or dental instruments, ophthalmic devices, wound treatments (bandages, sutures, cell scaffolds, bone cements, particles), appliances, implants, scaffolding, suturing material, valves, pacemaker, stents, catheters, rods, implants, fracture fixation devices, pumps, tubing, wiring, electrodes, contraceptive devices, feminine hygiene products, endoscopes, wound dressings and other devices, which come into contact with tissue, especially human tissue.

A. Fibrous and Particulate Materials

In one embodiment, the peptides are applied to a fibrous material, or are incorporated into a fibrous material or a coating on a fibrous material. These include would dressings, bandages, gauze, tape, pads, sponges, including woven and non-woven sponges and those designed specifically for dental or ophthalmic sugeries (See, e.g., U.S. Pat. Nos. 4,098,728; 4,211,277; 4,636,208; 5,180,375; and 6,711,879), paper or polymeric materials used as surgical drapes, disposable diapers, tapes, bandages, feminine products, sutures, and other fibrous materials. One of the advantages of the immobilized peptides is that they are not only antibacterial at the time of application, but help to minimize contamination by the materials after disposal.

Fibrous materials are also useful in cell culture and tissue engineering devices. Bacterial and fungal contamination are major problems in eukaryotic cell culture and this provides a safe and effective way to minimize or eliminate contamination of the cultures.

The peptides are also readily bound to particles, including nanoparticles, microparticles and millimeter beads, which have uses in a variety of applications including cell culture and drug delivery.

B. Implanted and Inserted Materials

The peptides can also be applied directly to, and coupled by ionic, covalent or hydrogen bonding to, or incorporated into, polymeric, metallic, or ceramic substrates, for examples, catheters, tubing, heart valves, drug pumps, orthopedic implants, and other devices inserted into, implanted in or applied to a patient.

Representative implantable materials include heart valves, pacemakers, stents, catheters including central venous catheters (“CVC”) and urinary catheters, ventricular assist devices, and bone repair devices including screws, plates, rivets, rods, bone cements, and prosthetics.

Studies demonstrate that high loading can be achieved by direct coupling of peptides to polyurethane and silicone, primary materials used for devices such as CVCs.

C. Coatings, Paints, Dips, Sprays

The peptides can also be added to paints and other coatings and filters to prevent mildew, bacterial comtamination, and in other applications where it is desirable to provide antimicrobial activity.

### EXAMPLES

The present invention will be further understood by reference to the following non-limiting examples.

### Example 1

### Antimicrobial Activity of an Immobilized Antimicrobial Peptide

Materials and Methods

Synthesis of Antimicrobial Peptide

The antimicrobial peptide, cystein-incorporating Cecropin-Melittin hybride peptide (KWKLFKKIGAVLKVLC-NH₂) (SEQ ID NO:5), was synthesize using fluorenylmethoxycarbonyl (Fmoc) chemistry using an Intavis Multipep Synthesizer (available from Intavis LLC).

NH₂-microparticles (TentaGel S-NH₂ resin, Anaspec. Cat. #22795) were used as the substrate to immobilize a cysteine-incorporating Cecropin-Melittin hybride peptide (KWKLFKKIGAVLKVLC-NH₂) (SEQ ID NO:5) via the tether N-[γ-maleimidobutyryl-oxy]succinimide ester. The number of free amino groups was quantified using the ninhydrin assay. Approximately 6.7 mg of microparticles were suspended in 1 mL of 1 M acetate buffer pH 5.0 containing 12.5 mg of ninhydrin (Sigma). The suspension was kept in boiling water for 15 min. After 15 min the sample was removed and 15 mL of an ethanol/water mixture (1/1, v/v) was added. The reaction mixture was allowed to cool to room temperature for 1 hour, away from light. Ninhydrin reacts with free amino groups and creates a blue water-soluble compound. The amount of free amino groups in the beads was spectrophotometrically determined by measuring the absorbance of the supernatant at 570 nm, after the 1 hour cooling time. Glycine was used as a reference material.

Coupling of the Peptide to Tether-Functionalized Beads

3.4 mg of sulfo-GMBS was reacted with 15 mg of NH₂-microparticles suspended in 0.5 mL PBS buffer having a pH of 7.4 at room temperature for two hours with mild agitation (vortex, 100 rpm), After two hours, the beads were centrifuged for two minutes at 2500 rpm and washed five times with 1 mL of PBS buffer. In the last wash, the beads were re-suspended in 0.5 mL PBS buffer and reacted with 5 mg of a cysteine-incorporating Cecropin-Melittin hybrid peptide (KWKLFKKIGAVLKVLC-NH2) (SEQ ID NO: 5), overnight, at room temperature with mild agitation (100 rpm). the beads were against washed 5 times with 0.5 mL PBS buffer and then re-suspended in 1 mL of PBS and kept at 4° C. overnight. The following morning the supernatant was removed, and the beads were washed 5 times with 1 mL of PBS buffer. The beads were re-suspended in 1 mL and stored at 4° C. The peptide immobilized in the beads was determined by the BCA assay (Sigma), using cecropin melittin as the standard. The amount of peptide bound to beads was determined indirectly from the difference between the initial total peptide exposed to the beads and the amount of peptide recovered in the several washes. The concentration of peptide bound to the beads was approximately 0.91 mg per 15 mg of beads, which corresponds to 0.060 mg of peptide per 72.7 mm² of bead surface area, assuming the bead is non-porous.

Antimicrobial Activity

The peptide conjugated beads were tested against Escherichia coli by incubating with 1×10⁷ cfu/ml K12 E. coli in CMHB which had been stained with 30 μM propidium iodide and 6 μM STYO9 stains from a standard Molecular probes LIVE/DEAD kit. As determined with a fluorescence microscope, 50% of the bacteria in solution were killed after one hour. To assess whether the killing effect was truly due to the immobilized peptide, the medium that was incubated with the beads was centrifuged at 3000 rpm for 2 minutes, the supernatant was removed, and the supernatant was inoculated with 1×10⁷ cfu/ml E. coli in CMHB for 1 hour. No killing was observed. This indicates that the immobilized peptide is the effective component against bacteria.

### Example 2

### Antimicrobial Peptides Immobilized on a Planar Surface Exhibit Antimicrobial Properties

A cysteine-incorporating Cecropin-Melittin hybrid peptide (KWKLFKKIGAVLKVLC-NH₂) (SEQ ID NO: 5) was immobilized on a commercial membrane with terminal amine groups (0.340 μmoles of NH₂ per cm², as determined by the picric acid assay) (Intavis product number 30.100), that is used for the solid state synthesis of peptides. The terminal amine groups of the membrane was reacted with the succinimide groups of sulfo-GMBS and in a subsequent step the maleimide groups of sulfo-GMBS was reacted with the thiol groups of the cysteine-incorporating peptide. The amount of peptide bound to the membrane was determined indirectly from the difference between the initial total peptide exposed to the beads and the amount of peptide recovered in the several washes. The quantity of immobilized peptide was approximately 2.0 mg per cm² of membrane. This peptide-conjugated membrane was tested for immobilized bactericidal activity against Escherichia coli ATCC 2592.

An overnight culture of a target bacteria in a growth medium such as Cation Adjusted Mueller Hinton Broth, was diluted to approximately 1×10⁵ cfu/ml in pH 7.4 phosphate Buffered Saline using a predetermined calibration between OD₆₀₀ and cell density. A 0.5 cm² sample of immobilized antimicrobial surface was added to 0.75 ml of the bacterial suspension. The sample was covered by the liquid and incubated at 37° C. with a sufficient amount of mixing so that the solid surface is seen to rotate through the liquid. After 1 hour of incubation, serial dilutions of the bacterial suspension were plated on agar plates and allowed to grow overnight for quantifying the viable cell concentration. Using this procedure, the peptide conjugated membrane produced a 4.2-log reduction of E. coli in solution over 1 h. Testing the amine-functionalized membrane without an antimicrobial peptide conjugated to it for immobilized bactericidal activity did not show a significant reduction in viable bacteria (<0.1 log reduction).

### Example 3

### Antimicrobial Peptides Immobilized on a Planar Surface Exhibit Antimicrobial Properties after more than 3 Weeks Storage in PBS through Repeated Challenges of Bacteria

Samples identical to those generated in Example 2 and stored at 4° C. in pH 7.4 PBS for more than three weeks. When this peptide-conjugated membrane was tested against for immobilized bactericidal activity against Escherichia coli, an average of a 1.8-log reduction of bacteria in solution occurred over 1 h. The samples were then removed from the testing solution, and placed in fresh PBS. Samples then underwent 10 minutes of ultrasonication, switched to fresh PBS, and underwent an additional 30 minutes of sonication. They were then rinsed and retested. The immobilized antibacterial activity, using the assay described in Example 2, of the washed samples was measured against Escherichia coli ATCC 25922, and an average of a 3.3-log reduction in bacteria occurred in 1 hour.

### Example 4

### Confirmation that Antimicrobial Activity does not Result from Leached Agent

A test was carried out to determine whether the samples used in Example 3 were non-leaching. An evaluation of the supernatant was used to show that the samples used in Example 3 were non-leaching during both rounds of killing before and after washing. At the end of the 1 hour incubation between the sample and a solution of bacteria described in Example 3, 0.4 ml of bacterial solution was removed. The 0.4 ml was centrifuged at 3000×g for 5 minutes to remove remaining bacteria. A sample of 0.2 ml of supernatant was removed and added to 0.05 ml of Escherichia coli ATCC 25922 at 5×10⁵ cfu/ml, giving a final concentration of 1×10⁵ cfu/ml, as in the standard antibacterial assay. This mixture was incubated at 37° C. with the same degree of mixing as in the immobilized bactericidal activity assay, and serial dilutions were plated at the end of 1 hour.

The supernatant from both the 1^{st} and 2^{nd} rounds of killing did not show a measurable amount of killing (<0.1-log reduction in viable bacteria). Because the surface demonstrated killing, but the supernatant above the surface does not demonstrate any killing, the immobilized antimicrobial surface is substantially non-leaching.

### Example 5

### Antimicrobial Peptides can be Covalently Immobilized into a Gel while Keeping Its Antimicrobial Properties

Dextran gels were prepared by UV crosslinking of dextran acrylate macomonomer. Dextran-acrylate with a degree of substitution of 23.3% (400 mg) (please see Ferreira et al., Biomaterials 2002, 23, 3957-3967 for details in preparation) was dissolved in PBS (1.8 ml) and Igracure (5 mg/ml, 250 μL) was gently mixed into the solution. Cross-linking of the solution was initiated by exposure UV-light over a 10 minute period. The resulting gel was cut into several disks (8 mm diameter) using a biopsy punch, and washed overnight in water. Prior to the functionalization reaction, each dextran disk was soaked in 95% ethanol for 20 minutes, shrinking the gel. Then, the shrunken gel was soaked in a solution of sodium periodate (5.3 mg/ml, 1 ml) in PBS, for 1 hour with mild agitation (vortex, 100 rpm). After this time the disk was washed (5 times) in PBS to remove an un-reacted sodium periodate. The disk was then placed in a solution of ethylene diamine dihydrochloride (66 mg/ml, 1 ml) and the reaction was allowed to continue for 1½ hours with mild agitation (vortex, 100 rpm). After this step the disk was rinsed thoroughly (5times) in PBS. A solution of sodium cyanoborohydride (15 mg/mL, 1 ml) was prepared in PBS and allowed to cool to room temperature for 10 minutes after mixing. The disk was allowed to react, without agitation, in the sodium cyanoborohydride solution for 30 minutes followed by thorough rinsing and overnight soaking in PBS. The functionalized gel was soaked in 95% ethanol for 20 minutes followed by soaking in a sulfo-GMBS (10 mg/ml, 0.4 ml) solution for 2 hours at room temperature, with mild agitation (vortex, 100 rpm). Excess sulfo-GMBS was removed by rinsing with PBS (5 times). The disk was then soaked again in 95% ethanol for 2 minutes followed by soaking in a cysteine-incorporating Cecropin-Melittin hybrid peptide (KWKLFKKIGAVLKVLC-NH2) (SEQ ID NO:5) solution (5 mg/ml) overnight, at room temperature, with mild agitation (vortex, 100 rpm). The disk was washed 10 times (0.5 ml, PBS), over a 2 day period, and the washings were kept for the determination of peptide released. BCA assay showed 3.22 mg of peptide was immobilized on the dextran disk.

When assayed for immobilized bactericidal activity, a gel functionalized with a cystein-incorporating Cecropin-Melittin hybrid peptide demonstrated a 2.9-log reduction in Escherichia coli ATCC 25922, whereas a gel without Cecropin-Melittin hybrid peptide did not display a significant reduction in viable bacteria (<0.1-log).

### Example 6

### The Orientation in the Covalent Immobilization of an Antimicrobial Peptide onto a Substrate is Important for Its Ultimate Biological Activity

To determine whether the orientation of the immobilized peptide is important for its bioactivity, a cysteine-incorporating Cecropin-Melittin hybrid peptide (KWKLFKKIGAVLKVLC-NH2) (SEQ ID NO:5) was immobilized with random orientation by coupling the multiple peptide amine groups to a membrane surface containing carboxylic groups. The following protocol was followed. A cellulose membrane (1×1 cm²) containing terminal amine groups was incubated with a solution of Methyl N-succinimidyl adipate (MSA, Pierce) (1.54 mg in 0.1 mL of a solution of DMSO in PBS pH 7.4 (1:9, v/v)) for 2 h, at room temperature. The membrane was then washed several times (5×1 mL) with PBS and incubated in phosphate buffer pH 9.5 (2 mL) overnight. After that time, the membrane was washed with PBS pH 7.4 (5×1 mL) and 0.1 M citrate buffer pH 7.5 (5×1 mL). The membrane was reacted with 0.5 mL of N-(3-dimethylaminopropyl)-N′-ethyl-carbodiimide hydrochloride (EDC) solution (4.8 mg/mL in 0.1 M sodium citrate buffer pH 5.0) for 30 minutes and afterwards washed with PBS (3×1 mL). The activated membrane was subsequently reacted with a cysteine-incorporating Cecropin-Melittin hybrid peptide (KWKLFKKIGAVLKVLC-NH2) (SEQ ID NO:5) peptide (5 mg in 1 mL of PBS), overnight, at room temperature, with mild agitation (100 rpm), and finally washed with PBS (10 times, 1 mL washes) and kept in PBS, 4° C., until use.

The results show that most of the terminal amine groups of the cellulose membrane did react with MSA. The content of amine groups was 0.340 μmol/cm² and 0.039 μmol/cm², before and after MSA reaction, respectively.

The terminal COOH groups of MSA were coupled with the terminal NH₂ groups of the antimicrobial peptide using EDC chemistry. The peptide immobilized on the membrane was determined by the BCA assay (Sigma), using a cysteine-incorporating Cecropin-Melittin hybrid peptide (KWKLFKKIGAVLKVLC-NH2) (SEQ ID NO: 5) as the standard. The amount of peptide bound to the membrane was determined from the difference between the initial total peptide exposed to the membrane and the amount of peptide recovered in the several washes.

The peptide content was 1.84±0.27 mg/cm² (n=2). The content of peptide per surface area was similar to the one immobilized using an oriented peptide (sulfo-GMBS chemistry) (1.75±0.36 mg per cm², n=3). Both oriented and non-oriented peptides with similar surface densities were evaluated for immobilized bactericidal activity against E. coli ATCC 25922. The oriented peptide produced a 3.0-log reduction in viable bacteria, whereas the non-oriented peptide produced only a 1.6-log reduction. This shows that the immobilization of an antimicrobial peptide in an oriented way creates a higher specific biological activity.

### Example 7

### Oriented Immobilized Antimicrobial Peptide has High Specific Activity

A cysteine-incorporating Cecropin-Melittin hybrid peptide (KWKLFKKIGAVLKVLC-NH2) (SEQ ID NO: 5) was immobilized to the amine presenting cellulose membrane with the sulfo-GMBS chemistry as described in Example 2, with the exception that the concentration of peptide in solution was varied. The concentration of peptide in solution during the immobilization step was varied from 0.125 mg/ml to 5.0 mg/ml. Samples were assayed for immobilized bactericidal activity as described in Example 2. When a concentration of 5 mg/ml was used during immobilization, the resulting surface produced a 2.0-log reduction of E. coli ATCC in 1 hour. However, when the concentration of peptide during immobilization was reduced to 0.125 mg/ml, a 1.8-log reduction still occurred, which is at a significantly lower density than the non-oriented peptide in Example 6. Thus, a greater immobilized bactericidal activity is achieved per mass of peptide used when the peptide is oriented (higher specific activity)

### Example 8

### The Immobilized Antimicrobial Peptide Surface is Substantially Non-Hemolytic

A cysteine-incorporating Cecropin-Melittin hybrid peptide (KWKLFKKIGAVLKVLC-NH2) (SEQ ID NO: 5) was immobilized to the amine presenting cellulose membrane with the sulfo-GMBS chemistry as described in Example 2, and the sample was tested to see if it was a substantially non-hemolytic surface. A stock of 10% washed pooled red blood cells (Rockland Immunochemicals Inc, Gilbertsville, Pa.) is diluted to 0.25% with a hemolysis buffer of 150 mM NaCl and 10 mM Tris at pH 7.0. A 0.5 cm2 antimicrobial sample is incubated with 0.75 ml of 0.25% red blood cell suspension for 1 hour at 37° C. The solid sample is removed and cells spun down at 6000 g, the supernatant removed, and the OD414 measured on a spectrophotometer. Total hemolysis is defined by diluting 10% of washed pooled red blood cells to 0.25% in sterile DI water and incubating for 1 hour at 37° C., and 0% hemolysis is defined by a suspension of 0.25% red blood cells in hemolysis buffer without a solid sample. The peptide immobilized sample produced only 4.95% hemolysis using this assay, demonstrating that the sample is a substantially non-hemolytic surface.

### Example 9

### Coupling of an Antimicrobial Peptide to Amidated Polymer Brushes

The brush monomer, aminoethyl methacrylate (AEMA), was placed in a buffered methanol/water solution along with azobisisobutyronitrile (AIBN). The solution was incubated at 70° C. above a vinyl presenting substrate for one hour. As the AEMA polymerized, vinyl units on the substrate surface were incorporated into the growing polymers chains, tethering these chains to the substrate. A schematic of the resulting material is shown in FIG. 2. Following the polymerization, the surface was rinsed repeatedly and then ultrasonicated in phosphate buffered saline to remove any ungrafted polymer chains. Samples were then dried and the thicknesses measured. Additional ultrasonication failed to further reduce film thickness, indicating that all remaining polymer was covalently attached to the substrate. Polymer composition was verified through IR spectroscopy.

After thickness and composition verification, a cysteine-incorporating Cecropin-Melittin hybrid peptide (KWKLFKKIGAVLKVLC-NH2) (SEQ ID NO:5) was immobilized on the surface using the sulfo-GMBS chemistry described in Example 1. Initial immobilization experiments with the polymer brush surface showed a greater than four fold increase in mass of immobilized peptide per surface area compared to planar substrates. All of the immobilized peptide is surface presented, dramatically increasing the effective AmP concentration. Optimization of polymer brush molecular weight and branching further increases effective surface concentration of immobilized peptide.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A composition comprising a substrate having immobilized thereon one or more antimicrobial peptides, wherein the antimicrobial peptides are uniformly tethered in a specified orientation.

2. The composition of claim 1 wherein the antimicrobial peptides are immobilized by bonds selected from the group consisting of covalent bonds, non-covalent bonds, and combinations of covalent and non-covalent bonds thereof.

3. The composition of claim 1 wherein the immobilized antimicrobial activity of the oriented peptides is greater than the immobilized antimicrobial activity of the same surface density and type of the peptides randomly tethered to the substrate without specified orientation.

4. The composition of claim 1 wherein the surface has immobilized antimicrobial activity at or below 0.2 mg/cm², more preferably at or below 0.1 mg/cm², even more preferably at or below 0.05 mg/cm², and most preferably at or below 0.01 mg/cm².

5. The composition of claim 1 wherein the peptides are oriented by specifically binding to their C-terminus.

6. The composition of claim 1 wherein the peptides are linear peptides.

7. The composition of claim 1 wherein the composition is substantially non-leaching and biocompatible.

8. The composition of claim 1 wherein the composition is substantially anti-fouling.

9. The composition of claim 1 wherein the composition is substantially non-cytotoxic.

10. The composition of claim 1 wherein the composition is substantially non-hemolytic.

11. The composition of claim 1 wherein the antimicrobial peptide sequence is between more than 9 and less than 150, more preferably less than 100, most preferably than 9-51, amino acids is length.

12. The composition of claim 1 wherein the antimicrobial peptide sequence is non-naturally occurring.

13. The composition of claim 1 wherein more than one peptide sequence is immobilized.

14. The composition of claim 1 wherein the immobilized antimicrobial activity of the oriented peptides is antibacterial.

15. The composition of claim 1 wherein the peptide is bound to the substrate by ionic binding.

16. The composition of claim 1 wherein the peptide is bound to the substrate by the interaction of strepavidin and biotin, polyhistidine-nickel chelate coupling, or salicylhydroxamic acid-phenyl boronic acid.

17. The composition of claim 1 wherein the surface of the substrate is modified through a gas-phase technique selected from the group consisting of plasma, corona discharge, flame treatment, UV/Ozone, UV and ozone only, aminolysis, hydrolysis, reduction, activation of alcohol chain ends with tosyl chloride and subsequent chemistry, graft copolymerisation of vinyl compounds by chemical initiation, or ion beam treatment in the presence of vinyl monomers.

18. The composition of claim 1 wherein the substrate surface is treated to introduce groups on the substrate surface, which can react with functional groups on the peptide, wherein the groups on the substrate are selected from the group consisting of hydroxyl, amine, halide, epoxide, activated ester, sulfhydryl, vinyl, and carboxylic acid groups.

19. The composition of claim 1 wherein thiol or amino groups in the peptides can react directly by conjugate addition reaction with unsaturated groups such as maleimides, vinyl sulfones, acrylamides and acrylates present in the substrate on the substrate.

20. The composition of claim 1 wherein the peptide is bound to the substrate by a functional group present in the peptide selected from the group consisting of amine, thiol, carbonyl, carboxyl, aldehyde, vinyl, phenyl, and alcohol.

21. The composition of claim 1 wherein one or more amine, alcohol or thiol groups on the peptide is reacted directly with a functional group on the surface of the substrate selected from the group consisting of isothiocyanate, acyl azide, N-hydroxysuccinimide ester, aldehyde, epoxide, anhydride, halides, sulphydryl, vinyl, and lactone.

22. The composition of claim 1 where one or more free amino, sulfhydryl or hydroxyl groups of the peptides are attached to a surface containing epoxide functional groups.

23. The composition of claim 1 comprising a tether or spacer molecule between the peptide and substrate.

24. The composition of claim 23 wherein the tether is hydrophilic polymer.

25. The composition of claim 24 wherein the tether is polyethylene glycol (PEG).

26. The composition of claim 23 wherein the peptide is coupled to the substrate with a homobifunctional sulfhydryl-reactive coupling agent.

27. The composition of claim 23 wherein the peptide is coupled to the substrate with a heterobifunctional sulfhydryl-reactive coupling agent.

28. The composition of claim 27 wherein the coupling agent is sulfo-GMBS.

29. The composition of claim 1 wherein a polymer is grafted onto the substrate and the peptides are covalently coupled to the polymer.

30. The composition of claim 29 wherein the polymer is crosslinked to form a gel.

31. The composition of claim 30 wherein the crosslinked polymer is Dextran.

32. The composition of claim 29 wherein the polymer is a polymer brush attached to the substrate.

33. The composition of claim 29 wherein the polymer is dendrimeric polymer attached to the substrate.

34. The composition of claim 29 wherein the polymer is synthesized by chemical vapor deposition.

35. The composition of claim 29 wherein the polymer is attached to a substrate formed of a material selected from the group consisting of silicone or polyurethane.

36. The composition of claim 1 wherein the peptide is attached to the substrate at a density of between 0.125 and 50 mg/cm².

37. The composition of claim 1 wherein the peptide is attached to the substrate at a density of greater than 0.5 mg/cm², more preferably 1 mg/cm², even more preferably 5 mg/cm², even more preferably 10 mg/cm², and most preferably greater than 25 mg/cm².

38. The composition of claim 1 wherein the antimicrobial activity remains for repeated uses with washing or storage for 21 days in organic or aqueous solvents between uses.

39. The composition of claim 1 wherein the substrate is a polymer, ceramic, or metal.

40. The composition of claim 39 wherein the substrate is in the form of an implantable or injectable device.

41. The composition of claim 40 wherein the device is selected from the group consisting of stents, catheters, tubing, needles, pacemakers, prosthetics, bone cement, screws, rivets, plates, valves, grafts, sensors, surgical instruments, and pumps.

42. The composition of claim 1 wherein the substrate is a tissue engineering or tissue culture support or matrix.

43. The composition of claim 1 wherein the substrate is fibrous.

44. The composition of claim 43 wherein the fibrous substrate is in the form of a device selected from the group consisting of gauze, pads, wound dressings, surgical drapes, surgical garments, diapers, and sponges.

45. The composition of claim 1 wherein the substrate is a membrane.

46. The composition of claim 1 wherein the substrate is in the form of nanoparticles, microparticles or beads.

47. The composition of claim 1 wherein the substrate further comprises one or more therapeutic, prophylactic, or diagnostic agents which are covalently tethered to the surface or optionally released independently of the immobilized antimicrobial peptide.

48. The composition of claim 47 wherein the therapeutic, prophylactic or diagnostic agent is selected from the group consisting of antiproliferative, cytostatic, or cytotoxic chemotherapeutic agents, antimicrobial agents, anti-inflammatory agents, growth factors, antithromobotic agents, and cell adhesion peptides.

49. The composition of claim 47 wherein the therapeutic, prophylactic or diagnostic agent is tethered to the substrate using a hydrolyzable linkage so that the agent is slowly released from the substrate.
